(19) United States
(12) Patent Application Publication
(10) Pub. No.: US 2026/0232290 A1
(43) Pub. Date: Aug. 13, 2026

(54) ULTRASONIC DIAGNOSTIC APPARATUS, IMAGE PROCESSING APPARATUS, METHOD, AND NON-TRANSITORY COMPUTER READABLE MEDIUM

(71) Applicants: CANON MEDICAL SYSTEMS CORPORATION, Tochigi (JP); Canon Kabushiki Kaisha, Tokyo (JP)

(72) Inventors: Hiroki TAKAHASHI, Nasushiobara (JP); Takeshi SATO, Nasushiobara (JP); Masashi USUMURA, Utsunomiya (JP); Shoya SASAKI, Taito (JP); Naoya IIZUKA, Yokohama (JP)

(73) Assignees: CANON MEDICAL SYSTEMS CORPORATION, Tochigi (JP); Canon Kabushiki Kaisha, Tokyo (JP)

(21) Appl. No.: 19/535,039

(22) Filed: Feb. 10, 2026

(30) Foreign Application Priority Data
Feb. 10, 2025 (JP) 2025-020153

(51) Int. Cl. A61B 8/08; A61B 8/00; A61B 8/06

(52) U.S. Cl. CPC A61B 8/0891; A61B 8/06; A61B 8/461; A61B 8/488; A61B 8/5223

(57) ABSTRACT

An ultrasonic diagnostic apparatus according to embodiments includes processing circuitry. The processing circuitry divides at least a part of an ultrasonic image into a plurality of regions. The processing circuitry determines whether each of the regions is a vascular region. The processing circuitry normalizes statistical information about blood flow in a region determined to be a vascular region. The processing circuitry synthesizes the normalized statistical information.

### CROSS-REFERENCE TO RELATED APPLICATIONS

This application is based upon and claims the benefit of priority from Japanese Patent Application No. 2025-020153, filed on Feb. 10, 2025; the entire contents of which are incorporated herein by reference.

### FIELD

Embodiments described herein relate generally to an ultrasonic diagnostic apparatus, an image processing apparatus, a method, and a non-transitory computer readable medium.

### BACKGROUND

Statistical information on the velocity of blood flow in microvessels distributed within a subject's tumor (formed within a tumor) is useful information for doctor's diagnosis. For example, the statistical difference in blood flow velocity within a tumor between hemangiomas, adenomas, and hepatocellular carcinomas (HCC) can be used to differentiate tumor grade. A method of distinguishing a malignant tumor in the lymph node by using the statistical difference in blood flow velocity between blood flow in vessels distributed in a non-malignant area in the lymph node and blood flow in vessels distributed in a malignant tumor has also been investigated in clinical practice. In this way, there is a method for measuring statistical velocity information of blood flow distributed within a subject to which a contrast medium is administered.

However, the methods described above require the administration of a contrast medium to the subject, resulting in a relatively long examination time and relatively low examination throughput.

### BRIEF DESCRIPTION OF THE DRAWINGS

FIG. 1 is a block diagram illustrating an example configuration of an ultrasonic diagnostic apparatus according to a first embodiment;

FIG. 2 is a diagram for explaining an example of processing executed by the ultrasonic diagnostic apparatus according to the first embodiment;

FIG. 3 is a diagram for explaining an example of processing executed by the ultrasonic diagnostic apparatus according to the first embodiment;

FIG. 4 is a diagram for explaining an example of processing executed by the ultrasonic diagnostic apparatus according to the first embodiment;

FIG. 5 is a diagram for explaining an example of processing executed by the ultrasonic diagnostic apparatus according to the first embodiment;

FIG. 6 is a flowchart illustrating an example of the flow of processing executed by the ultrasonic diagnostic apparatus according to the first embodiment;

FIG. 7 is a flowchart illustrating an example of the flow of processing executed by the ultrasonic diagnostic apparatus according to a first modification of the first embodiment;

FIG. 8 is a diagram for explaining an example of processing executed by the ultrasonic diagnostic apparatus according to the first modification of the first embodiment;

FIG. 9 is a flowchart illustrating an example of the flow of processing executed by the ultrasonic diagnostic apparatus according to a second embodiment;

FIG. 10 is a flowchart illustrating an example of the flow of processing executed by the ultrasonic diagnostic apparatus according to a third embodiment;

FIG. 11 is a diagram for explaining an example of processing executed by the ultrasonic diagnostic apparatus according to the third embodiment using a speckle tracking method;

FIG. 12 is a diagram for explaining an example of processing executed by the ultrasonic diagnostic apparatus according to the third embodiment using a speckle tracking method; and

FIG. 13 is a diagram illustrating an example configuration of an image processing apparatus according to a fourth embodiment.

### DETAILED DESCRIPTION

One of problems to be solved by embodiments disclosed herein and in the drawings is to obtain information about blood flow without using a contrast medium, that is, in a non-contrast manner. However, the problems to be solved by the embodiments disclosed herein and in the drawings are not limited to the above problem. Problems corresponding to the effects achieved by the configurations illustrated in the embodiments described below can also be considered as other problems.

An ultrasonic diagnostic apparatus according to embodiments includes processing circuitry. The processing circuitry divides at least a part of an ultrasonic image into a plurality of regions. The processing circuitry determines whether each of the regions is a vascular region. The processing circuitry normalizes statistical information about blood flow in a region determined to be a vascular region. The processing circuitry synthesizes the normalized statistical information.

An ultrasonic diagnostic apparatus, an image processing apparatus, a method, and a non-transitory computer readable medium according to each of embodiments and modifications will be described below with reference to the drawings. Hereinafter, parts denoted with the same reference signs are assumed to operate in the same way, and duplicated descriptions may be omitted as appropriate. The embodiments can be combined with other embodiments, modifications, or conventional technologies to the extent that there is no inconsistency in the contents of processing. Similarly, the modifications can be combined with embodiments, other modifications, or conventional technologies to the extent that there is no inconsistency in the contents of processing.

### First Embodiment

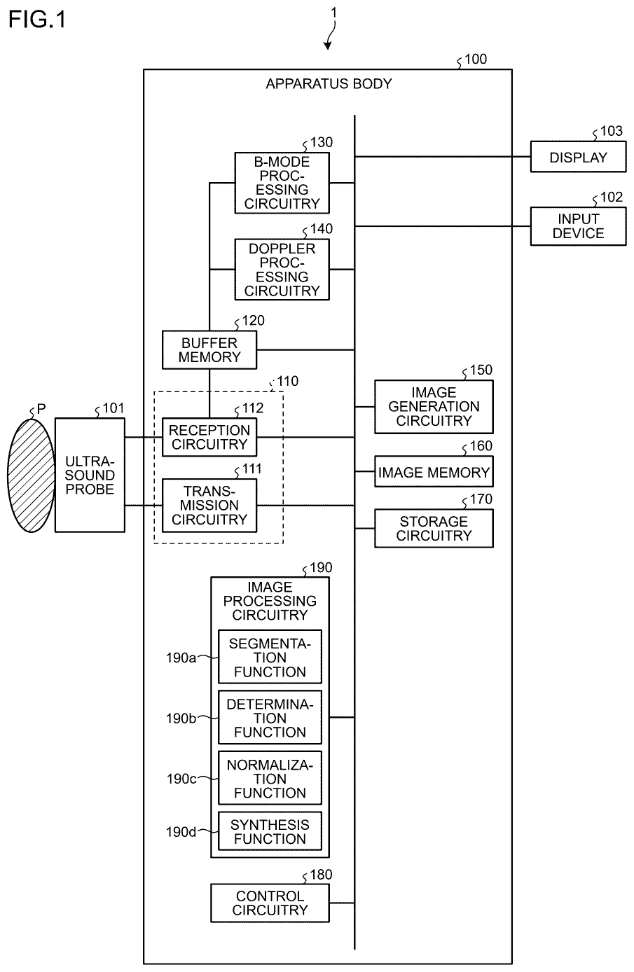

FIG. 1 is a block diagram illustrating an example configuration of an ultrasonic diagnostic apparatus 1 according to a first embodiment. As illustrated in FIG. 1, the ultrasonic diagnostic apparatus 1 according to the first embodiment includes an apparatus body 100, an ultrasound probe 101, an input device 102, and a display 103.

The ultrasound probe 101 has, for example, a plurality of elements (piezoelectric transducer elements, piezoelectric elements). These elements generate ultrasonic waves based on drive signals supplied by transmission circuitry 111 of transmission/reception circuitry 110 of the apparatus body 100. Specifically, the elements generate an ultrasonic wave having a waveform corresponding to a transmission drive voltage when a voltage (transmission drive voltage) is applied by the transmission circuitry 111. The waveform of the transmission drive voltage indicated by the drive signal is the waveform of the voltage applied to the elements. In other words, the ultrasound probe 101 transmits an ultrasonic wave according to the magnitude of the applied transmission drive voltage. The ultrasound probe 101 receives a reflected wave from a subject P, converts the received reflected wave into a reflected wave signal, which is an electrical signal, and outputs the reflected wave signal to the apparatus body 100. The ultrasound probe 101 has, for example, a matching layer on the elements and a backing material that prevents the propagation of ultrasonic waves from the elements to the back. The ultrasound probe 101 is detachably connected to the apparatus body 100.

When ultrasonic waves are transmitted from the ultrasound probe 101 to the subject P, the transmitted ultrasonic waves are reflected one after another at the acoustic impedance discontinuous surface in the body tissue of the subject P, and are received as reflected waves by the elements of the ultrasound probe 101. The amplitude of the received reflected waves depends on the difference in acoustic impedance at the discontinuous surface at which ultrasonic waves are reflected. When the transmitted ultrasonic pulse is reflected at a surface of a moving object, such as moving blood flow or heart wall, the reflected wave undergoes a frequency shift depending on a velocity component with respect to the direction of ultrasonic transmission of the moving object due to the Doppler effect. The ultrasound probe 101 then outputs the reflected wave signal to reception circuitry 112 of the transmission/reception circuitry 110 described below.

The ultrasound probe 101 is detachable from the apparatus body 100. When a two-dimensional region within the subject P is scanned (two-dimensional scanning), the operator connects, for example, a 1D array probe with a plurality of elements in a row as the ultrasound probe 101 to the apparatus body 100. Types of the 1D array probe include linear, convex, and sector ultrasound probes. When a three-dimensional region within the subject P is scanned (three-dimensional scanning), the operator connects, for example, a mechanical 4D probe or 2D array probe as the ultrasound probe 101 to the apparatus body 100. The mechanical 4D probe is capable of two-dimensional scanning using a plurality of elements arranged in a row, like a 1D array probe, and capable of three-dimensional scanning by swinging a plurality of elements at a predetermined angle (swing angle). The 2D array probe is capable of three-dimensional scanning with a plurality of elements arranged in a matrix and capable of two-dimensional scanning by focusing and transmitting ultrasonic waves.

In the present embodiment, the ultrasound probe 101 capable of executing a transmit aperture synthesis method or a plane wave compound method is used.

The input device 102 is implemented, for example, by input means such as a mouse, a keyboard, buttons, panel switches, a touch command screen, a foot switch, a trackball, and a joystick. The input device 102 accepts various setting requests from the operator (e.g., doctor) of the ultrasonic diagnostic apparatus 1 and transmits the accepted setting requests to the apparatus body 100. For example, the input device 102 accepts an instruction from the operator to set a region of interest (ROI) in a blood flow image displayed on the display 103 (region-of-interest setting instruction), and transmits the accepted region-of-interest setting instruction to the apparatus body 100. For example, the operator inputs a region-of-interest setting instruction to the input device 102 to set a region of interest in an area where the operator wants to grasp information (e.g., velocity information) about blood flow in the blood vessels (microvessels) formed in the tumor visualized in the blood flow image.

The display 103, for example, displays a graphical user interface (GUI) for the operator of the ultrasonic diagnostic apparatus 1 to input various setting requests using the input device 102, or displays an ultrasonic image based on ultrasonic image data generated in the apparatus body 100. The display 103 is implemented by a liquid crystal monitor, an organic light emitting diode (OLED) monitor, or the like. The display 103 is an example of a display unit.

The apparatus body 100 generates an ultrasonic image (ultrasonic image data) based on reflected wave signals transmitted from the ultrasound probe 101. The apparatus body 100 can generate two-dimensional ultrasonic image data based on reflected wave signals corresponding to a two-dimensional region of the subject P transmitted from the ultrasound probe 101. The apparatus body 100 can generate three-dimensional ultrasonic image data based on reflected wave signals corresponding to a three-dimensional region of the subject P transmitted from the ultrasound probe 101. As illustrated in FIG. 1, the apparatus body 100 includes transmission/reception circuitry 110, a buffer memory 120, B-mode processing circuitry 130, Doppler processing circuitry 140, image generation circuitry 150, an image memory 160, storage circuitry 170, control circuitry 180, and image processing circuitry 190.

The transmission/reception circuitry 110 allows the ultrasound probe 101 to transmit ultrasonic waves and allows the ultrasound probe 101 to receive reflected waves of the ultrasonic waves, under control by the control circuitry 180. In other words, the transmission/reception circuitry 110 executes scanning through the ultrasound probe 101. The scanning is also referred to as scan, ultrasonic scan, or ultrasonic scanning. The transmission/reception circuitry 110 is an example of a transmitter/receiver. The transmission/reception circuitry 110 includes the transmission circuitry 111 and the reception circuitry 112. The transmission circuitry 111 is an example of a transmitter, and the reception circuitry 112 is an example of a receiver.

The transmission circuitry 111 supplies a drive signal to the ultrasound probe 101 under control by the control circuitry 180 to allow the ultrasound probe 101 to transmit an ultrasonic wave. The transmission circuitry 111 has a rate pulser generation circuit, a transmission delay circuit, and a transmission pulser. When a two-dimensional region within the subject P is scanned, the transmission circuitry 111 allows the ultrasound probe 101 to transmit an ultrasound beam for scanning the two-dimensional region. When a three-dimensional region within the subject P is scanned, the transmission circuitry 111 allows the ultrasound probe 101 to transmit an ultrasound beam for scanning the three-dimensional region.

The rate pulser generation circuit repeatedly generates a rate pulse for forming a transmission ultrasonic wave (transmission beam) at a predetermined pulse repetition frequency (PRF) under control by the control circuitry 180. As the rate pulse passes through the transmission delay circuit, a voltage is applied to the transmission pulser with different transmission delay times. For example, the transmission delay circuit applies, to each rate pulse generated by the rate pulser generation circuit, a transmission delay time for each element that is necessary to focus the ultrasonic waves generated by the ultrasound probe 101 into a beam and determine the transmission directivity. The transmission pulser supplies a drive signal (drive pulse) to the ultrasound probe 101 at a timing based on the rate pulse. In other words, the transmission pulser applies a voltage with a waveform indicated by the drive signal (transmission drive voltage) to the ultrasound probe 101 at the timing based on the rate pulse. The transmission delay circuit adjusts the transmission direction of ultrasonic waves from the element surface as desired by varying the transmission delay time applied to each rate pulse.

The drive pulse is transmitted from the transmission pulser to the element in the ultrasound probe 101 via a cable, and then converted from an electrical signal to mechanical vibration in the element. In other words, the element vibrates mechanically when a voltage is applied to the element. The ultrasonic wave generated by this mechanical vibration is transmitted inside the living body (inside the subject P). Here, ultrasonic waves with different transmission delay times for each element are focused and propagate in a predetermined direction.

The transmission circuitry 111 has a function capable of instantaneously changing a transmission frequency, a transmission drive voltage, and the like to execute a predetermined scanning sequence, under control by the control circuitry 180. In particular, the changing of a transmission drive voltage is realized by a linear amplifier type transmission circuitry that can instantaneously switch the value of the transmission drive voltage, or by a mechanism that electrically switches a plurality of power supply units. The transmission frequency is, for example, the center frequency of the transmitted ultrasonic wave.

The reflected wave of the ultrasonic wave transmitted by the ultrasound probe 101 reaches the element inside the ultrasound probe 101 and is then converted from mechanical vibration to an electrical signal (reflected wave signal) in the element, and the reflected wave signal is input to the reception circuitry 112. The reception circuitry 112 includes a preamplifier, an analog to digital (A/D) converter, a quadrature detection circuit, and the like, and performs various processing on the reflected wave signal transmitted from the ultrasound probe 101 to generate reflected wave data. The reception circuitry 112 then stores the generated reflected wave data into the buffer memory 120.

The preamplifier amplifies the reflected wave signal for each channel and performs gain adjustment (gain correction). The A/D converter converts the gain-corrected reflected wave signal to a digital signal by A/D conversion of the gain-corrected reflected wave signal. The quadrature detection circuit converts the reflected wave signal converted to a digital signal into an in-phase signal (I signal, I: In-phase) and a quadrature signal (Q signal, Q: Quadrature-phase) in the baseband band. The quadrature detection circuit then stores the I and Q signals (IQ signals) as reflected wave data into the buffer memory 120.

The reception circuitry 112 performs various processing on the reflected wave signal transmitted from the ultrasound probe 101 to generate reflected wave data. The reception circuitry 112 then stores the generated reflected wave data into the buffer memory 120.

The reception circuitry 112 generates two-dimensional reflected wave data from a two-dimensional reflected wave signal transmitted from the ultrasound probe 101. The reception circuitry 112 also generates three-dimensional reflected wave data from a three-dimensional reflected wave signal transmitted from the ultrasound probe 101.

Here, in the present embodiment, the ultrasound probe 101 and the transmission/reception circuitry 110 are capable of collecting a plurality of pieces of reflected wave data along a time series by the transmit aperture synthesis method or the plane wave compound method.

In the present embodiment, the ultrasonic diagnostic apparatus 1 can perform various processing in real time. For example, the ultrasound probe 101 transmits the reflected wave signals for one frame one after another to the reception circuitry 112. Each time the reception circuitry 112 receives the reflected wave signals for one frame transmitted from the ultrasound probe 101, the reception circuitry 112 generates reflected wave data for one frame from the reflected wave signals for one frame. Each time the reception circuitry 112 generates reflected wave data for one frame, the reception circuitry 112 stores the reflected wave data for one frame into the buffer memory 120.

The buffer memory 120 is a memory that temporarily stores therein reflected wave data generated by the transmission/reception circuitry 110. For example, the buffer memory 120 is configured to store therein reflected wave data for a predetermined number of frames. When reflected wave data for one frame is newly generated by the reception circuitry 112 while the buffer memory 120 stores therein a predetermined number of frames of reflected wave data, the buffer memory 120 discards the reflected wave data for one frame generated earliest and stores therein the newly generated reflected wave data for one frame, under the control of the reception circuitry 112. For example, the buffer memory 120 is implemented by a semiconductor memory element such as a random access memory (RAM) or a flash memory.

The B-mode processing circuitry 130 reads reflected wave data from the buffer memory 120, performs various signal processing on the read reflected wave data, and outputs the reflected wave data subjected to various signal processing as B-mode data to the image generation circuitry 150. The B-mode processing circuitry 130 is implemented, for example, by a processor. The B-mode processing circuitry 130 is an example of a B-mode processing unit.

For example, each time reflected wave data for one frame is newly stored into the buffer memory 120, the B-mode processing circuitry 130 reads the reflected wave data for one frame newly stored in the buffer memory 120. The B-mode processing circuitry 130 then performs various signal processing on the read reflected wave data for one frame to newly generate B-mode data for one frame. Each time the B-mode processing circuitry 130 generates B-mode data for one frame, the B-mode processing circuitry 130 outputs the newly generated B-mode data for one frame to the image generation circuitry 150. An example of various signal processing executed by the B-mode processing circuitry 130 will be described below.

For example, the B-mode processing circuitry 130 performs quadrature detection, logarithmic amplification and envelope detection processing, and the like on the reflected wave data read from the buffer memory 120 to generate B-mode data representing the signal intensity (amplitude intensity) for each sample point in terms of brightness of luminance. The B-mode processing circuitry 130 then outputs the generated B-mode data to the image generation circuitry 150.

The Doppler processing circuitry 140 reads reflected wave data from the buffer memory 120, performs various signal processing on the read reflected wave data, and outputs the reflected wave data subjected to various signal processing as Doppler data to the image generation circuitry 150. The Doppler processing circuitry 140 is implemented, for example, by a processor. The Doppler processing circuitry 140 is an example of a Doppler processing unit.

For example, each time reflected wave data for one frame is newly stored into the buffer memory 120, the Doppler processing circuitry 140 reads the reflected wave data for one frame newly stored in the buffer memory 120. The Doppler processing circuitry 140 then performs various signal processing on the read reflected wave data for one frame to newly generate Doppler data for one frame. Each time the Doppler processing circuitry 140 generates Doppler data for one frame, the Doppler processing circuitry 140 outputs the newly generated Doppler data for one frame to the image generation circuitry 150. An example of various signal processing executed by the Doppler processing circuitry 140 will be described below.

For example, the Doppler processing circuitry 140 extracts motion information of a moving object (blood flow, tissue, contrast medium echo components, etc.) based on the Doppler effect from the reflected wave data by frequency analysis of the reflected wave data read from the buffer memory 120, and generates Doppler data indicating the extracted motion information. For example, the Doppler processing circuitry 140 extracts average velocity, average variance, average power, and the like over multiple points, as motion information of a moving object, and generates Doppler data indicating the extracted motion information of the moving object. The Doppler processing circuitry 140 outputs the generated Doppler data to the image generation circuitry 150.

Using the functions of the Doppler processing circuitry 140 described above, the ultrasonic diagnostic apparatus 1 can execute a color Doppler method, also called a color flow mapping (CFM) method. In the color flow mapping method, transmission and reception of ultrasonic waves are performed multiple times on a plurality of scanning lines. In the color flow mapping method, a moving target indicator (MTI) filter is applied to a data sequence at the same location to suppress a signal originating from stationary or slow-moving tissue (clutter signal) and extract a signal originating from blood flow (blood flow signal) from the data sequence at the same location. In the color flow mapping method, blood flow information such as blood flow velocity (average velocity), blood flow variance (average variance), and blood flow power (average power) is estimated from the blood flow signal. The Doppler processing circuitry 140 outputs color Doppler data indicating blood flow information estimated by the color flow mapping method to the image generation circuitry 150.

The Doppler processing circuitry 140 according to the present embodiment uses, as an MTI filter, an adaptive MTI filter that changes its coefficients according to an input signal. For example, the Doppler processing circuitry 140 uses an adaptive MTI filter called “eigenvector regression filter”. The “eigenvector regression filter”, which is an adaptive MTI filter using eigenvectors, is hereinafter referred to as “eigenvector MTI filter”.

The eigenvector MTI filter calculates eigenvectors from a correlation matrix and calculates, from the calculated eigenvectors, coefficients used in the clutter component suppressing process. This method is an application of the techniques used in principal component analysis, Karhunen-Loeve transform, and the eigenspace method.

The Doppler processing circuitry 140 according to the first embodiment using the eigenvector MTI filter calculates the correlation matrix of a first segmented region described below, from a data sequence of consecutive reflected wave data at the same location (same sample point). The Doppler processing circuitry 140 then calculates the eigenvalues of the correlation matrix and the eigenvectors corresponding to the eigenvalues. The Doppler processing circuitry 140 then calculates, as a filter matrix that suppresses a clutter component, a matrix that reduces the rank of the matrix in which the eigenvectors are arranged based on the magnitude of each eigenvalue.

The Doppler processing circuitry 140 then uses the filter matrix to identify a data sequence from which the clutter component is suppressed and the blood flow signal originating from blood flow is extracted, from the data sequence of consecutive reflected wave data at the same location (same sample point). The Doppler processing circuitry 140 then estimates blood flow information by performing calculations such as autocorrelation operations using the identified data sequence. The Doppler processing circuitry 140 then outputs color Doppler data indicating the estimated blood flow information to the image generation circuitry 150.

The B-mode processing circuitry 130 and the Doppler processing circuitry 140 can process both two-dimensional reflected wave data and three-dimensional reflected wave data.

The image generation circuitry 150 generates various ultrasonic image data (ultrasonic images) from B-mode data, second harmonic components, and third harmonic components output from the B-mode processing circuitry 130, and Doppler data and color Doppler data output from the Doppler processing circuitry 140. For example, the image generation circuitry 150 is implemented by a processor.

For example, the image generation circuitry 150 generates two-dimensional B-mode image data representing the intensity of the reflected wave in terms of luminance, from the two-dimensional B-mode data generated by the B-mode processing circuitry 130. The image generation circuitry 150 generates two-dimensional Doppler image data or two-dimensional color Doppler image data visualizing motion information or blood flow information from the two-dimensional Doppler data or color Doppler data generated by the Doppler processing circuitry 140. The two-dimensional Doppler image data visualizing motion information and the two-dimensional color Doppler image data visualizing blood flow information are velocity image data (velocity image), variance image data (variance image), power image data (power image), or image data (image) of a combination of these.

In addition to color Doppler image data for color display, the image generation circuitry 150 can also generate, for example, gray-scale power image data in which the luminance is varied in gray scale according to the value of power.

The color Doppler image data and the gray-scale power image data visualizing blood flow information are also referred to as blood flow image data (blood flow image).

Here, the image generation circuitry 150 generally converts (scan-converts) a scanning line signal sequence of ultrasonic scanning into a video-format scanning line signal sequence as typified by television or the like, and generates ultrasonic image data for display. For example, the image generation circuitry 150 generates ultrasonic image data for display by performing coordinate transformation on data output from the B-mode processing circuitry 130 and the Doppler processing circuitry 140 according to the mode of ultrasonic scanning by the ultrasound probe 101. In addition to scan conversion, the image generation circuitry 150 may also perform various image processing, such as image processing to regenerate an average image of luminance using a plurality of image frames after scan conversion (smoothing process) or image processing using a differential filter in an image (edge enhancement process). The image generation circuitry 150 may also combine text information, scales, body marks, and the like for various parameters into the ultrasonic image data.

Furthermore, the image generation circuitry 150 generates three-dimensional B-mode image data by performing coordinate transformation on three-dimensional B-mode data generated by the B-mode processing circuitry 130. The image generation circuitry 150 also generates three-dimensional Doppler image data by performing coordinate transformation on three-dimensional Doppler data generated by the Doppler processing circuitry 140. In other words, the image generation circuitry 150 generates “three-dimensional B-mode image data and three-dimensional Doppler image data” as “three-dimensional ultrasonic image data (volume data)”. The image generation circuitry 150 then performs various rendering processes on the volume data to generate various two-dimensional image data for displaying the volume data on the display 103.

The rendering processes performed by the image generation circuitry 150 include, for example, a process of generating MPR image data from the volume data using a multi planer reconstruction (MPR) method. The rendering processes performed by the image generation circuitry 150 include, for example, a volume rendering (VR) process to generate two-dimensional image data reflecting three-dimensional information. The image generation circuitry 150 is an example of an image generation unit.

The B-mode data and the Doppler data are ultrasonic image data before the scanning conversion process, and the data generated by the image generation circuitry 150 is ultrasonic image data for display after the scanning conversion process. The B-mode data and the Doppler data are also referred to as raw data.

The image memory 160 is a memory that stores therein various image data generated by the image generation circuitry 150. The image memory 160 also stores therein data generated by the B-mode processing circuitry 130 and the Doppler processing circuitry 140. The B-mode data and the Doppler data stored in the image memory 160, for example, can be invoked by the operator after diagnosis and become ultrasonic image data for display via the image generation circuitry 150. For example, the image memory 160 is implemented by a semiconductor memory element such as a random access memory (RAM) or a flash memory, a hard disk, or an optical disk.

The storage circuitry 170 stores therein a control program for scanning (ultrasonic transmission and reception), image processing, and display processing, as well as diagnostic information (e.g., patient ID, doctor's findings, etc.) and various data such as diagnostic protocols and various body marks. The storage circuitry 170 is also used to archive therein data stored in the image memory 160, if necessary. For example, the storage circuitry 170 is implemented by a semiconductor memory element such as a flash memory, a hard disk, or an optical disk.

The control circuitry 180 controls the entire processing of the ultrasonic diagnostic apparatus 1. Specifically, the control circuitry 180 controls the processing in the transmission circuitry 111, the reception circuitry 112, the B-mode processing circuitry 130, the Doppler processing circuitry 140, and the image generation circuitry 150, based on various setting requests input from the operator through the input device 102, and various control programs and various data read from the storage circuitry 170. The control circuitry 180 also controls the display 103 to display ultrasonic images based on various ultrasonic image data for display stored in the image memory 160. For example, the control circuitry 180 controls the display 103 to display a B-mode image based on the B-mode image data or a color Doppler image based on the color Doppler image data. The control circuitry 180 also controls the display 103 to superimpose a color Doppler image on a B-mode image. The control circuitry 180 also controls the ultrasonic scanning by controlling the ultrasound probe 101 through the transmission/reception circuitry 110.

The control circuitry 180 is an example of a display control unit or a control unit. The control circuitry 180 is implemented, for example, by a processor.

The image processing circuitry 190 performs various image processing. As illustrated in FIG. 1, the image processing circuitry 190 includes a segmentation function 190a, a determination function 190b, a normalization function 190c, and a synthesis function 190d. The segmentation function 190a is, for example, an example of a segmentation unit. The determination function 190b is, for example, an example of a determination unit. The normalization function 190c is, for example, an example of a normalization unit. The synthesis function 190d is, for example, an example of a synthesis unit.

The image processing circuitry 190 is implemented, for example, by a processor. In this case, each of the processing functions 190a to 190d described above is stored in the storage circuitry 170 in the form of a computer program executable by a computer. The image processing circuitry 190 reads each computer program stored in the storage circuitry 170 and executes the read computer program to implement each processing function corresponding to the computer program. In other words, the image processing circuitry 190 has each processing function illustrated in FIG. 1 in a state in which each computer program is read.

The image processing circuitry 190 may be configured as a combination of a plurality of independent processors, each processor executing a computer program to implement each processing function. The processing functions of the image processing circuitry 190 may be distributed or integrated into single or a plurality of processing circuitry. The processing functions of the image processing circuitry 190 may be implemented by a mixture of hardware such as circuitry and software. Although an example in which each computer program corresponding to each processing function is stored in the single storage circuitry 170 is described here, each computer program may be distributed and stored in a plurality of storage circuitry. For example, each computer program corresponding to each processing function may be distributed and stored in a plurality of storage circuitry, and the image processing circuitry 190 may read and execute each computer program from the storage circuitry.

The term “processor” as used in the description refers to, for example, circuitry such as a central processing unit (CPU), a graphics processing unit (GPU), an application specific integrated circuit (ASIC), or a programmable logic device (e.g., a simple programmable logic device (SPLD), a complex programmable logic device (CLPD), or a field programmable gate array (FPGA)). The processor reads a computer program stored in the storage circuitry 170 and executes the read computer program to implement the function. Instead of storing a computer program in the storage circuitry 170, the computer program may be embedded directly into the circuitry of the processor. In this case, the processor reads and executes the computer program embedded in the circuitry to implement the function. Each processor in the present embodiment is not limited to a case where each processor is configured as a single circuit. A single processor may be configured by combining a plurality of independent circuits to implement its functions. Furthermore, a plurality of circuitry in FIG. 1 (e.g., B-mode processing circuitry 130, Doppler processing circuitry 140, image generation circuitry 150, control circuitry 180, and image processing circuitry 190) may be integrated into a single processor to implement its functions. In other words, the B-mode processing circuitry 130, the Doppler processing circuitry 140, the image generation circuitry 150, the control circuitry 180, and the image processing circuitry 190 may be integrated into single processing circuitry implemented by a processor. The transmission/reception circuitry 110, the B-mode processing circuitry 130, the Doppler processing circuitry 140, the image generation circuitry 150, the control circuitry 180, and the image processing circuitry 190 may be integrated into single processing circuitry that includes a processor.

The overall configuration of the ultrasonic diagnostic apparatus 1 according to the first embodiment has been described above.

In the first embodiment, the ultrasonic diagnostic apparatus 1 executes the processing described below so that information about blood flow can be obtained without using a contrast medium, that is, in a non-contrast manner.

For example, in the present embodiment, the ultrasound probe 101 and the transmission/reception circuitry 110 collect a plurality of pieces of reflected wave data along a time series by the transmit aperture synthesis method or the plane wave compound method. The ultrasound probe 101 and the transmission/reception circuitry 110 are, for example, examples of a collection unit. The Doppler processing circuitry 140 and the image generation circuitry 150 generate gray-scale two-dimensional power image data (power image) one after another along a time series as blood flow image data (blood flow image) from the collected reflected wave data. The Doppler processing circuitry 140 and the image generation circuitry 150 are, for example, examples of a generation unit. The generated power image data is stored in the image memory 160. The reflected wave data used to generate such power image data is stored in the buffer memory 120. In other words, the reflected wave data corresponding to the generated power image data is stored in the buffer memory 120. A plurality of pieces of reflected wave data along a time series including the reflected wave data corresponding to the generated power image data are also stored in the buffer memory 120. In the first embodiment, each time gray-scale two-dimensional power image data for one frame is generated, the ultrasonic diagnostic apparatus 1 executes the processing described later, using the generated power image data and a plurality of pieces of reflected wave data along a time series including the reflected wave data corresponding to the power image data, so that information about blood flow can be obtained in a non-contrast manner.

In the following description, it is assumed that the operator has already input a region-of-interest setting instruction to the input device 102 to set a region of interest in the area where the operator wants to grasp information about the velocity of a flow of blood (blood flow) through the microvessels formed in the tumor visualized in the gray-scale power image data displayed on the display 103. It is also assumed that the control circuitry 180 of the apparatus body 100 has already set a region of interest in the gray-scale power image data based on the region-of-interest setting instruction. Once a region of interest is set in the power image data, the control circuitry 180 sets (superimposes) a region of interest of the same shape at the same location in the image space on the power image data newly generated one after another. The power image data is image data based on reflected wave data obtained from reflected wave signals from blood cells of the subject P.

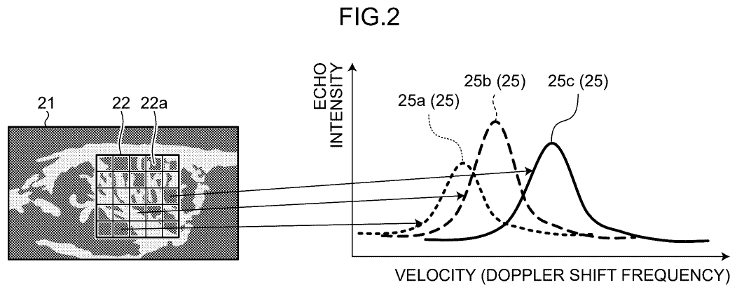

FIGS. 2 to 5 are diagrams for explaining an example of processing executed by the ultrasonic diagnostic apparatus 1 according to the first embodiment. First, the segmentation function 190a acquires the newly generated gray-scale two-dimensional power image data 21 for one frame illustrated in FIG. 2 from the image memory 160. The segmentation function 190a then divides a region of interest 22 set (superimposed) on the power image data 21 into a plurality of regions (local regions) 22a. In the example in FIG. 2, the segmentation function 190a divides the region of interest 22, which has the shape of a square with all four sides of equal length, into 16 (4×4) regions 22a. In this way, the segmentation function 190a divides at least a part of the power image data 21, which is ultrasonic image data, into the plurality of regions 22a. In various processing described below, processing is performed for each of the regions 22a. Therefore, when a region is divided into the plurality of regions 22a such that the number of pixels constituting one region 22a is one, the various processing is performed pixel by pixel.

The determination function 190b then determines whether the plurality of regions 22a are vascular regions. Here, the vascular region is a region in which blood vessels are visualized. An example of a method for determining whether a region is a vascular region in the first embodiment will be described. For example, the determination function 190b performs a binarization process on each of the plurality of regions 22a of the power image data 21. Specifically, the determination function 190b generates binarized image data by setting “1” to a pixel having a pixel value equal to or greater than a predetermined threshold value and “0” to a pixel having a pixel value smaller than the predetermined threshold value for each of a plurality of pixels constituting the region 22a. If the number of pixels having a pixel value of “1” among one or more pixels constituting the region 22a is equal to or greater than a predetermined number, the determination function 190b determines that the region 22a is a vascular region. On the other hand, if the number of pixels having a pixel value of “1” among one or more pixels constituting the region 22a is less than the predetermined number, the determination function 190b determines that the region 22a is not a vascular region. The determination function 190b performs the above processing for each region 22a.

If the proportion (ratio) of the number of pixels having a pixel value of “1” to the number of all pixels constituting the region 22a is equal to or greater than the predetermined threshold value, the determination function 190b may determine that the region 22a is a vascular region. The determination function 190b may also determine that the region 22a is not a vascular region if the proportion described above is smaller than the predetermined threshold value.

Then, the normalization function 190c first calculates a Doppler frequency spectrum for the region 22a determined to be a vascular region. The normalization function 190c calculates a Doppler frequency spectrum for each of the regions 22a determined to be vascular regions. The Doppler frequency spectrum is, for example, a curve with echo intensity (luminance) on the vertical axis and Doppler shift frequency or blood flow velocity based on Doppler shift frequency on the horizontal axis. In other words, the Doppler frequency spectrum indicates the relationship between echo intensity and blood flow velocity. The Doppler frequency spectrum is, for example, the Doppler spectrum of the region 22a determined to be a vascular region. The Doppler frequency spectrum is, for example, an example of statistical information about blood flow.

An example of the method for calculating a Doppler frequency spectrum according to the first embodiment will be described. For example, the normalization function 190c identifies the power image data of the region 22a determined to be a vascular region, from all of the power image data 21 stored in the image memory 160. The normalization function 190c then identifies the reflected wave data of the region 22a used to generate the identified power image data (the power image data of the region 22a determined to be a vascular region) from a plurality of pieces of reflected wave data along a time series stored in the buffer memory 120. The normalization function 190c then acquires a plurality of pieces of reflected wave data of the region 22a along a time series including the identified reflected wave data of the region 22a, from a plurality of pieces of reflected wave data along a time series stored in the buffer memory 120. Here, a plurality of pieces of reflected wave data of the region 22a along a time series are a plurality of pieces of reflected wave data of the region 22a aligned in the frame direction.

The normalization function 190c then calculates the Doppler frequency spectrum using a plurality of pieces of reflected wave data of the region 22a aligned in the frame direction, using a known technique. For example, the normalization function 190c calculates the Doppler frequency spectrum for each region 22a using a technique similar to the technique described in Japanese Patent Application Laid-open No. 2016-153005. Japanese Patent Application Laid-open No. 2016-153005 discloses, for example, a technique that acquires moving object information (blood flow information) of blood flow in a sample volume set in reflected wave data by performing frequency analysis using a Fast Fourier Transform (FFT) method, and generates a Doppler waveform based on the acquired blood flow information. In this way, Japanese Patent Application Laid-open No. 2016-153005 discloses a technique for acquiring blood flow information along a time series in a region of interest by performing frequency analysis between frames on reflected wave data in the region of interest for a plurality of frames. In the first embodiment, for example, the normalization function 190c calculates the Doppler frequency spectrum by using the region 22a as a sample volume and performing Fourier analysis in the frame direction on a plurality of pieces of reflected wave data (a plurality of pieces of reflected wave data aligned in the frame direction along a time series) of the region 22a determined to be a vascular region. Since the region 22a is used as a sample volume, the normalization function 190c acquires blood flow information (e.g., blood flow velocity) in a predetermined direction and calculates the Doppler frequency spectrum based on this blood flow information in the predetermined direction.

In the example illustrated in FIG. 2, three regions 22a are determined to be vascular regions. In this case, the normalization function 190c calculates a Doppler frequency spectrum 25a, a Doppler frequency spectrum 25b, and a Doppler frequency spectrum 25c respectively from the three regions 22a, as illustrated in the example in FIG. 2. The Doppler frequency spectrum 25a, the Doppler frequency spectrum 25b, and the Doppler frequency spectrum 25c may be referred to as “Doppler frequency spectrum 25” unless they are described separately.

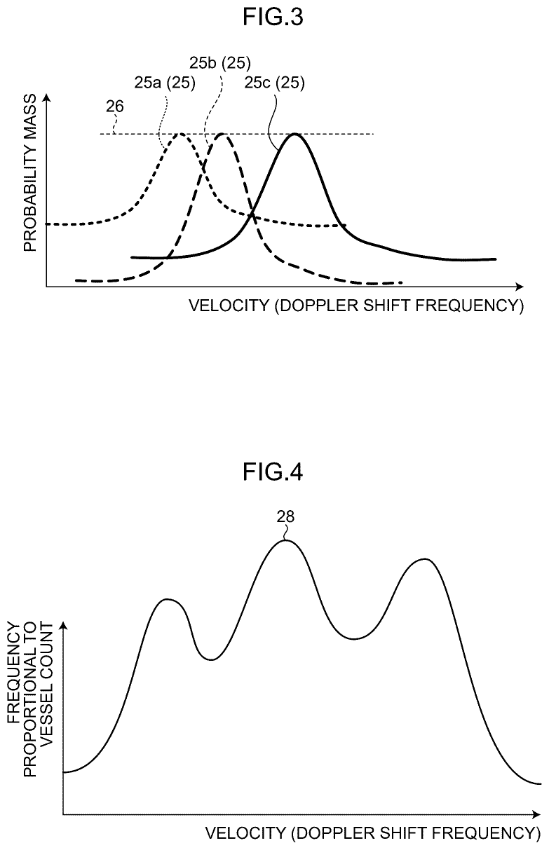

The normalization function 190c then normalizes the Doppler frequency spectrum 25. For example, as illustrated in FIG. 3, at least one of the Doppler frequency spectrum 25a, the Doppler frequency spectrum 25b, and the Doppler frequency spectrum 25c is moved along the vertical axis direction so that the peaks (peak values) in the vertical axis direction of the Doppler frequency spectrum 25a, the Doppler frequency spectrum 25b, and the Doppler frequency spectrum 25c are matched. In the example in FIG. 3, at least one of the Doppler frequency spectrum 25a, the Doppler frequency spectrum 25b, and the Doppler frequency spectrum 25c is moved along the vertical axis direction so that the peak values of the Doppler frequency spectrum 25a, the Doppler frequency spectrum 25b, and the Doppler frequency spectrum 25c are matched to a predetermined value on the vertical axis (the value corresponding to the chain line 26). In this way, the normalization function 190c corrects the Doppler frequency spectrum 25 based on the echo intensity. The normalization function 190c also normalizes the difference in echo intensity between the regions 22a determined to be vascular regions, that is, the difference in echo intensity between blood vessels. As a result, the vertical axis is considered as probability mass or probability density. In other words, the vertical axis is converted from echo intensity to probability mass or probability density. In this way, the normalization function 190c calculates the normalized Doppler frequency spectrum 25, which is a curve with probability mass or probability density on the vertical axis and Doppler shift frequency or blood flow velocity based on Doppler shift frequency on the horizontal axis.

The synthesis function 190d then synthesizes a plurality of normalized Doppler frequency spectra 25. For example, the synthesis function 190d synthesizes the normalized Doppler frequency spectrum 25a, the normalized Doppler frequency spectrum 25b, and the normalized Doppler frequency spectrum 25c. As a result, the synthesis function 190d generates a spectrum 28, which is a curve with frequency proportional to the number of blood vessels (vessel count) on the vertical axis and Doppler shift frequency or blood flow velocity based on Doppler shift frequency on the horizontal axis, as illustrated in FIG. 4. The spectrum 28 indicates the statistical distribution of vessel count with respect to blood flow velocity. The spectrum 28 is, for example, an example of synthetic statistical information. The vertical axis may be vessel count.

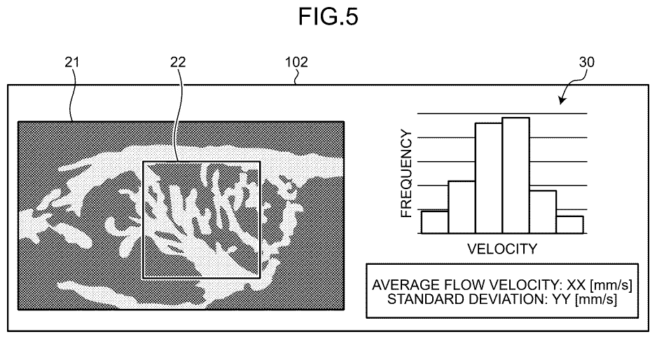

The synthesis function 190d then generates, from the spectrum 28, a histogram 30 (see FIG. 5) with frequency proportional to vessel count on the vertical axis and blood flow velocity based on Doppler shift frequency on the horizontal axis. The histogram 30 indicates the statistical distribution of vessel count with respect to blood flow velocity. The synthesis function 190d also calculates the average flow velocity (see FIG. 5), which is the average of the blood flow velocities of a plurality of vessels, from the spectrum 28 or the histogram 30. The synthesis function 190d also calculates the standard deviation (see FIG. 5) of blood flow velocities of a plurality of vessels from the spectrum 28 or the histogram 30. In this way, the synthesis function 190d calculates the histogram 30, the average flow velocity and the standard deviation of the blood flow velocities of a plurality of vessels, from the spectrum 28. The histogram 30, the average flow velocity and the standard deviation of blood flow velocities of a plurality of vessels are information about the blood flow velocity of microvessels distributed in a tumor and are useful for doctor's diagnosis. Therefore, according to the first embodiment, information about blood flow can be obtained in a non-contrast manner. The histogram 30, the average flow velocity and the standard deviation of blood flow velocities of a plurality of vessels are, for example, examples of statistical information for display. The histogram 30 is, for example, an example of a statistical graph. The average flow velocity and the standard deviation of blood flow velocities of a plurality of vessels are, for example, examples of statistics about blood vessels.

The control circuitry 180 then displays an image based on the power image data 21 with the region of interest 22 superimposed thereon, the histogram 30, and the average flow velocity and the standard deviation on the display 103, as illustrated in FIG. 5. Therefore, according to the first embodiment, information about blood flow can be presented to the operator.

The control circuitry 180 may display the spectrum 28 on the display 103. The spectrum 28 is also information about blood flow.

The synthesis function 190d may also calculate the mode of blood flow velocities of a plurality of vessels from the spectrum 28 or the histogram 30. The synthesis function 190d may also calculate the median of blood flow velocities of a plurality of vessels from the spectrum 28 or the histogram 30. The synthesis function 190d may also calculate the variance of blood flow velocities of a plurality of vessels from the spectrum 28 or the histogram 30. The synthesis function 190d may also calculate the skewness of blood flow velocities of a plurality of vessels from the spectrum 28 or the histogram 30. The synthesis function 190d may also calculate the kurtosis of blood flow velocities of a plurality of vessels from the spectrum 28 or the histogram 30. The synthesis function 190d may calculate at least one of the average flow velocity, standard deviation, mode, median, variance, skewness, and kurtosis of blood flow velocities of a plurality of vessels. The mode, median, variance, skewness, and kurtosis of blood flow velocities of a plurality of vessels are information about the blood flow velocity of microvessels distributed in a tumor and are useful for doctor's diagnosis. Thus, even in this case, information about blood flow can be obtained in a non-contrast manner. The mode, median, variance, skewness, and kurtosis of blood flow velocities of a plurality of vessels are, for example, examples of statistical information for display. The mode, median, variance, skewness, and kurtosis of blood flow velocities of a plurality of vessels are, for example, examples of statistics about blood vessels. The skewness is, for example, an example of a third-order moment. The kurtosis is, for example, an example of a fourth-order moment.

The control circuitry 180 may then display at least one calculated statistic among a plurality of statistics including mode, median, variance, skewness, and kurtosis of blood flow velocities of a plurality of vessels on the display 103. Also in this case, information about blood flow can be presented to the operator.

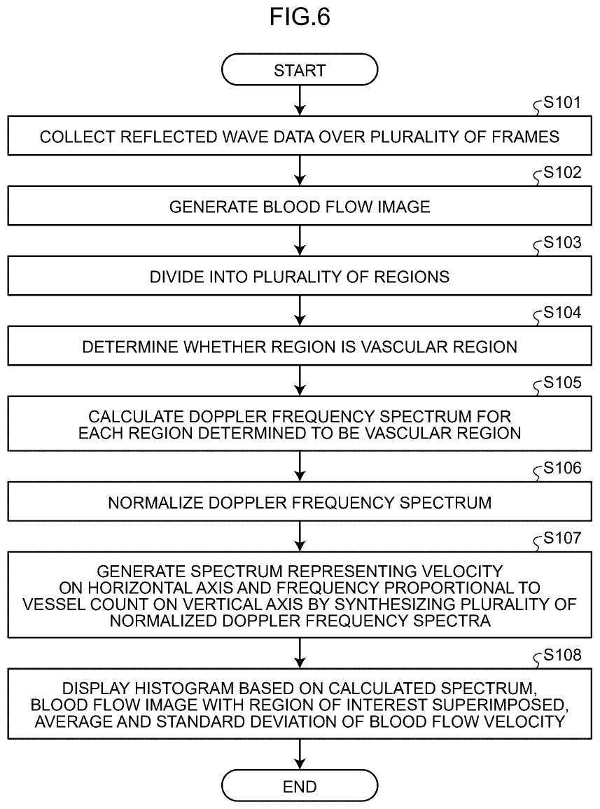

The flow of processing executed by the ultrasonic diagnostic apparatus 1 will now be described. FIG. 6 is a flowchart illustrating an example of the flow of processing executed by the ultrasonic diagnostic apparatus 1 according to the first embodiment.

As illustrated in FIG. 6, the ultrasound probe 101 and the transmission/reception circuitry 110 start collecting a plurality of pieces of reflected wave data along a time series over a plurality of frames, by the transmit aperture synthesis method or the plane wave compound method (step S101).

The Doppler processing circuitry 140 and the image generation circuitry 150 then start generating gray-scale two-dimensional power image data 21 one after another along a time series, as blood flow image data, from the collected reflected wave data (step S102).

Here, a plurality of processing from the processing at step S103 to the processing at step S108 described below are executed each time the power image data 21 is generated at step S102.

The segmentation function 190a then divides the region of interest 22 set in the power image data 21 into a plurality of the regions 22a (step S103). The determination function 190b then determines whether the plurality of regions 22a are vascular regions (step S104).

The normalization function 190c then calculates the Doppler frequency spectrum 25 for each of the regions 22a determined to be vascular regions (step S105). The normalization function 190c then normalizes the Doppler frequency spectrum 25 (step S106).

The synthesis function 190d then synthesizes a plurality of normalized Doppler frequency spectra 25 to generate the spectrum 28, which is a curve with frequency proportional to vessel count on the vertical axis and Doppler shift frequency or blood flow velocity based on Doppler shift frequency on the horizontal axis (step S107).

The synthesis function 190d then calculates the histogram 30, the average flow velocity and the standard deviation of blood flow velocities of a plurality of vessels from the spectrum 28, and the control circuitry 180 displays an image based on the power image data 21 with the region of interest 22 superimposed thereon, the histogram 30, as well as the average flow velocity and the standard deviation on the display 103 (step S108).

The ultrasonic diagnostic apparatus 1 does not necessarily execute the processing at step S108 in the process illustrated in FIG. 6.

The ultrasonic diagnostic apparatus 1 according to the first embodiment has been described above. According to the first embodiment, as described above, information about blood flow can be obtained in a non-contrast manner.

### First Modification

The ultrasonic diagnostic apparatus 1 according to a first modification of the first embodiment will now be described. The first embodiment describes a case where the determination function 190b determines for each region 22a whether the region 22a is a vascular region. In the first modification, the determination function 190b acquires (extracts) segmented vascular regions from the power image data 21 by performing morphology analysis on the power image data 21. The morphology analysis yields vascular regions having continuity.

The first embodiment describes a case where the ultrasonic diagnostic apparatus 1 performs various processing using the region 22a determined to be a vascular region. In the first modification, the ultrasonic diagnostic apparatus 1 performs processing similar to that of the first embodiment using a vascular region instead of the region 22a determined to be a vascular region. In the description of the ultrasonic diagnostic apparatus 1 according to the first modification, the points different from the first embodiment will be mainly described, and the description of the configuration similar to the first embodiment may be omitted.

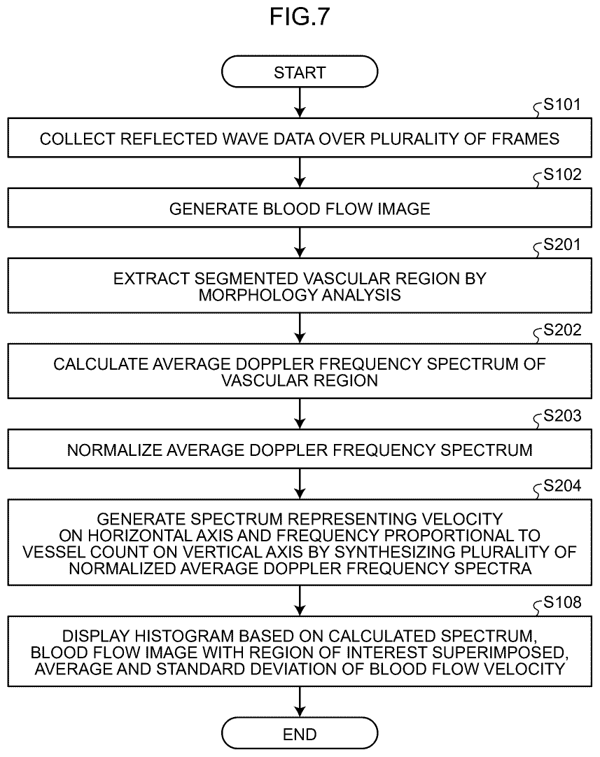

The flow of processing executed by the ultrasonic diagnostic apparatus 1 according to the first modification will be described. FIG. 7 is a flowchart illustrating an example of the flow of processing executed by the ultrasonic diagnostic apparatus 1 according to the first modification of the first embodiment.

The processing at step S101, the processing at step S102, and the processing at step S108 illustrated in FIG. 7 are the same as or similar to the processing at step S101, the processing at step S102, and the processing at step S108 according to the first embodiment illustrated in FIG. 6. A plurality of processing from the processing at step S201 to the processing at step S108 described below are executed each time the power image data 21 is generated at step S102.

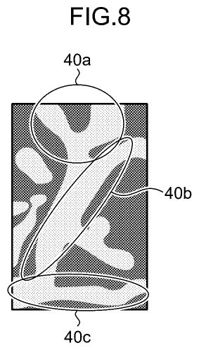

The determination function 190b acquires (extracts) segmented vascular regions from the power image data 21 by performing morphology analysis on the power image data 21 (step S201). FIG. 8 is a diagram for explaining an example of processing executed by the ultrasonic diagnostic apparatus 1 according to the first modification. FIG. 8 illustrates a part of the power image data 21. The determination function 190b acquires a vascular region depicted in a frame 40a, a vascular region depicted in a frame 40b, and a vascular region depicted in a frame 40c from the power image data 21, as illustrated in FIG. 8. In this way, the determination function 190b acquires vascular regions by morphology analysis on a spatial distribution based on power image data. The determination function 190b according to the first modification is, for example, an example of an acquisition unit.

The normalization function 190c then calculates, for each vascular region, the average Doppler frequency spectrum of the vascular region (step S202). An example of a method for calculating the average Doppler frequency spectrum will be described in detail. For example, the normalization function 190c divides the vascular region into a plurality of regions. Then, for each of the regions, the Doppler frequency spectrum is calculated in the same manner as in the first embodiment. As a result, a plurality of Doppler frequency spectra corresponding to a plurality of regions are calculated for each vascular region. The normalization function 190c then calculates the average of a plurality of Doppler frequency spectra as the average Doppler frequency spectrum.

The average Doppler frequency spectrum is, for example, a curve with echo intensity (luminance) on the vertical axis and Doppler shift frequency or blood flow velocity based on Doppler shift frequency on the horizontal axis. In other words, the average Doppler frequency spectrum indicates the relationship between echo intensity and blood flow velocity. The average Doppler frequency spectrum is, for example, the Doppler spectrum of a vascular region. The average Doppler frequency spectrum is, for example, an example of statistical information about blood flow.

The normalization function 190c then normalizes the average Doppler frequency spectrum for each vascular region (step S203). For example, the normalization function 190c first performs first normalization on the average Doppler frequency spectrum by dividing the echo intensity indicated by the average Doppler frequency spectrum by the area of the vascular region (intensity of reflected data/area of vascular region). As the area of the vascular region increases, the echo intensity increases. Thus, as a result of the first normalization, all average Doppler frequency spectra are treated as being obtained from the vascular regions with the same area.

The normalization function 190c then performs normalization (second normalization) on the average Doppler frequency spectrum subjected to the first normalization, based on the echo intensity, in the same manner as in the first embodiment. In other words, as a result of the second normalization, the peaks of a plurality of average Doppler frequency spectra are matched in the vertical axis direction. As a result, the vertical axis is considered as probability mass or probability density. In other words, the vertical axis is converted from echo intensity to probability mass or probability density. In this way, the normalization function 190c calculates the normalized average Doppler frequency spectrum, which is a curve with probability mass or probability density on the vertical axis and Doppler shift frequency or blood flow velocity based on Doppler shift frequency on the horizontal axis.

The synthesis function 190d then synthesizes a plurality of average Doppler frequency spectra subjected to the second normalization (step S204). As a result, the synthesis function 190d generates a spectrum, which is a curve with frequency proportional to the number of blood vessels (vessel count) on the vertical axis and Doppler shift frequency or blood flow velocity based on Doppler shift frequency on the horizontal axis, in the same manner as in the first embodiment. This spectrum indicates the statistical distribution of vessel count with respect to blood flow velocity. The spectrum is, for example, an example of synthetic statistical information.

Then, at step S108, the synthesis function 190d and the control circuitry 180 execute the following processing. For example, the synthesis function 190d generates, from the spectrum, a histogram with frequency proportional to vessel count on the vertical axis and blood flow velocity based on Doppler shift frequency on the horizontal axis, in the same manner as in the first embodiment. The synthesis function 190d also calculates, from the spectrum or the histogram, the average flow velocity, which is the average of blood flow velocities of a plurality of vessels, and the standard deviation of blood flow velocities of a plurality of vessels, in the same manner as in the first embodiment. The histogram, the average flow velocity and the standard deviation of blood flow velocities of a plurality of vessels are information about the blood flow velocity of microvessels distributed in a tumor and are useful for doctor's diagnosis. Therefore, according to the first modification, information about blood flow can be obtained in a non-contrast manner, in the same manner as in the first embodiment.

The control circuitry 180 then displays an image based on the power image data 21 with the region of interest 22 superimposed thereon, the histogram, as well as the average flow velocity and the standard deviation on the display 103, in the same manner as in the first embodiment. Therefore, according to the first modification, information about blood flow can be presented to the operator in the same manner as in the first embodiment.

The ultrasonic diagnostic apparatus 1 according to the first modification has been described above. According to the first modification, information about blood flow can be obtained in a non-contrast manner, in the same manner as in the first embodiment.

### Second Modification

The ultrasonic diagnostic apparatus 1 according to a second modification of the first embodiment will now be described. The first embodiment describes a case where the synthesis function 190d generates the spectrum 28 each time the power image data 21 is generated. In other words, in the first embodiment, the synthesis function 190d generates the spectrum 28 at a certain moment. In the second modification, the synthesis function 190d averages a plurality of spectra 28 for a predetermined period of time. For example, the synthesis function 190d averages a plurality of spectra 28 for a period of time equivalent to one heartbeat. As a result, a new spectrum can be obtained. The ultrasonic diagnostic apparatus 1 then performs processing similar to that of the first embodiment, using the new spectrum instead of the spectrum 28. For example, the synthesis function 190d generates a histogram as well as statistical information for display about blood flow velocity such as average flow velocity and standard deviation, from the new spectrum, in the same manner as in the first embodiment.

According to the second modification, information about blood flow can be obtained in a non-contrast manner, in the same manner as in the first embodiment.

### Second Embodiment

The ultrasonic diagnostic apparatus 1 according to a second embodiment will now be described. The first embodiment describes a case where the region 22a is used as a sample volume and the normalization function 190c acquires blood flow information (e.g., blood flow velocity) in a predetermined direction and calculates the Doppler frequency spectrum based on this blood flow information in the predetermined direction. However, the accuracy (precision) of the Doppler frequency spectrum is not always good if the predetermined direction does not match the main direction of blood flow (the direction in which blood mainly flows). Therefore, in the second embodiment, the ultrasonic diagnostic apparatus 1 corrects the echo intensity of the Doppler frequency spectrum according to the angle formed by the predetermined direction described above and the main direction of blood flow. In other words, the ultrasonic diagnostic apparatus 1 performs so-called angular correction to the Doppler frequency spectrum according to the angle formed by the predetermined direction described above and the main direction of blood flow. To illustrate with an example, letting the angle formed by the predetermined direction described above and the main direction of blood flow be θ, the ultrasonic diagnostic apparatus 1 performs angular correction by multiplying the Doppler frequency spectrum by COSθ. The angular correction is not limited to this. By performing angular correction to the Doppler frequency spectrum, the ultrasonic diagnostic apparatus 1 can obtain a Doppler frequency spectrum obtained from blood flow information in the direction in which blood mainly flows. The Doppler frequency spectrum thus obtained is referred to as velocity spectrum (velocity spectrum in the main direction) in the second embodiment.

In the description of the ultrasonic diagnostic apparatus 1 according to the second embodiment, the points different from the first embodiment will be mainly described, and the description of the configuration similar to the first embodiment may be omitted.

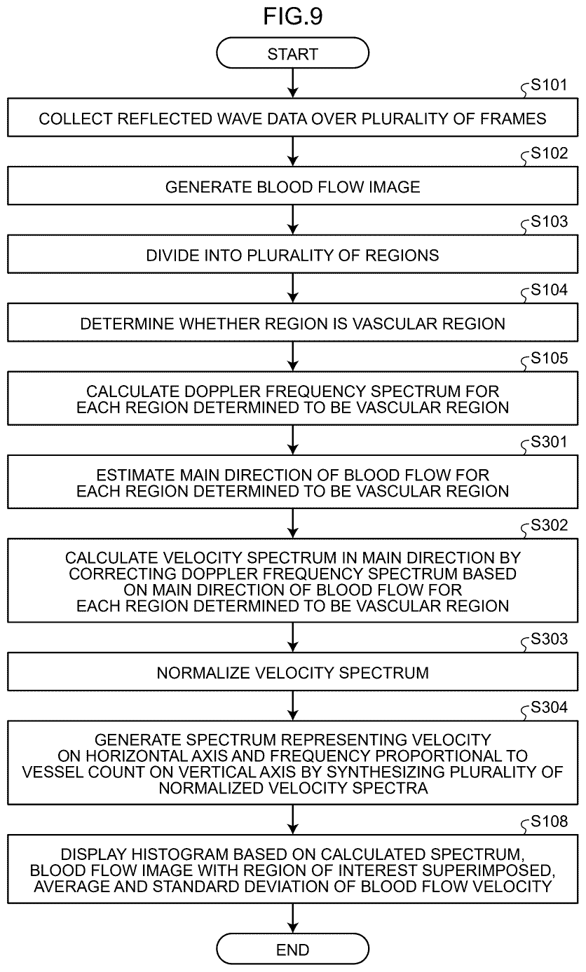

The flow of processing executed by the ultrasonic diagnostic apparatus 1 according to the second embodiment will be described. FIG. 9 is a flowchart illustrating an example of the flow of processing executed by the ultrasonic diagnostic apparatus 1 according to the second embodiment.

The processing at each of steps S101 to S105 and S108 illustrated in FIG. 9 is the same as or similar to the processing at each of steps S101 to S105 and S108 according to the first embodiment illustrated in FIG. 6. A plurality of processing from the processing at step S103 to the processing at step S108 illustrated in FIG. 9 are executed each time the power image data 21 is generated at step S102.

As illustrated in FIG. 9, the normalization function 190c estimates the main direction of blood flow through the vessel for each of the regions 22a determined to be vascular regions (step S301). For example, the normalization function 190c estimates the main direction of blood flow by analyzing the power image data of the region 22a determined to be a vascular region in the frame direction. To illustrate with a specific example, the normalization function 190c uses a speckle tracking method or a “multi-angle Doppler method” to estimate the main direction of blood flow in the region 22a determined to be a vascular region from a plurality of pieces of power image data of the regions 22a adjacent in the frame direction.

The normalization function 190c then calculates the velocity spectrum in the main direction by correcting the Doppler frequency spectrum 25 based on the main direction of blood flow, for each of the regions 22a determined to be vascular regions (step S302). At step S302, the normalization function 190c calculates the Doppler frequency spectrum (velocity spectrum in the main direction) obtained from the blood flow information in the direction in which blood mainly flows, by performing angular correction to the Doppler frequency spectrum 25 according to the angle formed by the predetermined direction described above and the main direction of blood flow. Such a velocity spectrum is, for example, an example of statistical information about blood flow.

The normalization function 190c then normalizes the velocity spectrum in the main direction by a method similar to the method for normalizing the Doppler frequency spectrum 25 at step S106 of the first embodiment (step S303).

The synthesis function 190d then synthesizes the normalized velocity spectrum in the main direction by a method similar to the method for synthesizing the normalized Doppler frequency spectrum 25 at step S107 of the first embodiment (step S304). The processing at step S304 generates a spectrum, which is a curve with frequency proportional to vessel count on the vertical axis and Doppler shift frequency or blood flow velocity based on Doppler shift frequency on the horizontal axis. The spectrum indicates the statistical distribution of vessel count with respect to blood flow velocity. The spectrum is also information about blood flow with good accuracy. The spectrum is, for example, an example of synthetic statistical information.

Then, at step S108, the synthesis function 190d and the control circuitry 180 execute the following processing. For example, the synthesis function 190d generates, from the spectrum, a histogram with frequency proportional to vessel count on the vertical axis and blood flow velocity based on Doppler shift frequency on the horizontal axis, in the same manner as in the first embodiment. The synthesis function 190d also calculates, from the spectrum or the histogram, the average flow velocity, which is the average of blood flow velocities of a plurality of vessels, and the standard deviation of blood flow velocities of a plurality of vessels, in the same manner as in the first embodiment. The histogram, the average flow velocity and the standard deviation of blood flow velocities of a plurality of vessels are information about the blood flow velocity of microvessels distributed in a tumor and are useful for doctor's diagnosis. Therefore, according to the second embodiment, information about blood flow with good accuracy can be obtained in a non-contrast manner, in the same manner as in the first embodiment.

The control circuitry 180 then displays an image based on the power image data 21 with the region of interest 22 superimposed thereon, the histogram, as well as the average flow velocity and the standard deviation on the display 103, in the same manner as in the first embodiment. Therefore, according to the second embodiment, information about blood flow with good accuracy can be presented to the operator.

The ultrasonic diagnostic apparatus 1 according to the second embodiment has been described above. According to the second embodiment, information about blood flow with good accuracy can be obtained in a non-contrast manner.

### Third Embodiment

The ultrasonic diagnostic apparatus 1 according to a third embodiment will now be described. The ultrasonic diagnostic apparatus 1 according to the third embodiment performs the processing of generating an absolute velocity spectrum described below, rather than the Doppler frequency spectrum 25.

In the description of the ultrasonic diagnostic apparatus 1 according to the third embodiment, the points different from the first embodiment will be mainly described, and the description of the configuration similar to the first embodiment may be omitted.

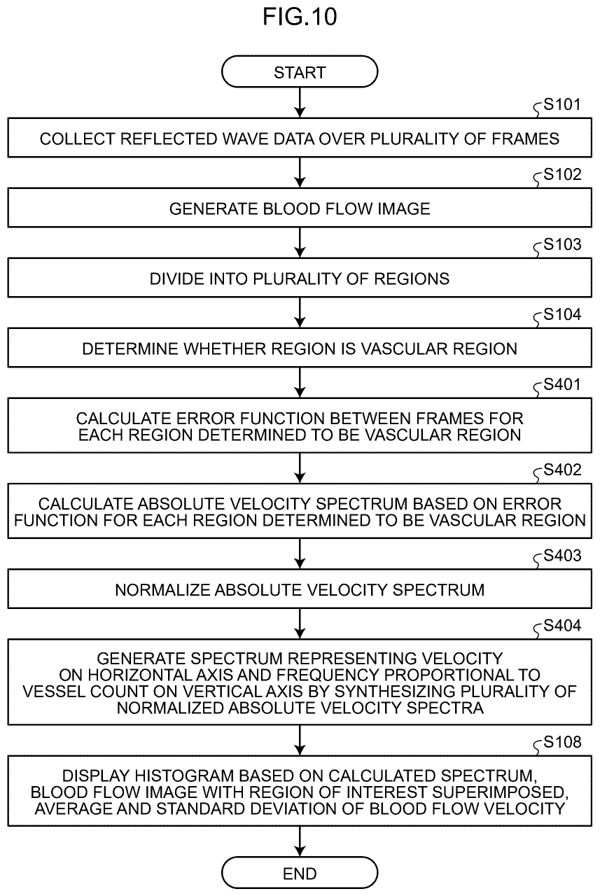

The flow of processing executed by the ultrasonic diagnostic apparatus 1 according to the third embodiment will be described. FIG. 10 is a flowchart illustrating an example of the flow of processing executed by the ultrasonic diagnostic apparatus 1 according to the third embodiment.

The processing at each of steps S101 to S104 and S108 illustrated in FIG. 10 is the same as or similar to the processing at each of steps S101 to S104 and S108 according to the first embodiment illustrated in FIG. 6. A plurality of processing from the processing at step S103 to the processing at step S108 illustrated in FIG. 10 are executed each time the power image data 21 is generated at step S102.

As illustrated in FIG. 10, the normalization function 190c calculates an error function between a plurality of pieces of the power image data 21 adjacent in the frame direction, for each local blood vessel location (in this case, for each of the regions 22a determined to be vascular regions) (step S401). For example, the normalization function 190c calculates the error function between a plurality of pieces of power image data 21 adjacent in the frame direction according to a speckle tracking method or a block matching method.

For example, the normalization function 190c searches between two pieces of power image data 21 adjacent in the frame direction for the image data most similar to the image data at a local blood vessel location in one of the power image data 21, while shifting the search position in the entire region in the other power image data 21. Specifically, the normalization function 190c calculates the similarity between the image data at the local blood vessel location in one of the power image data 21 and the image data at the search position in the other power image data 21 while shifting the search position. As a result, a plurality of similarities are calculated. The normalization function 190c then treats the image data at the search position corresponding to the highest similarity among a plurality of similarities as the image data at the local blood vessel location after the elapse of one frame.

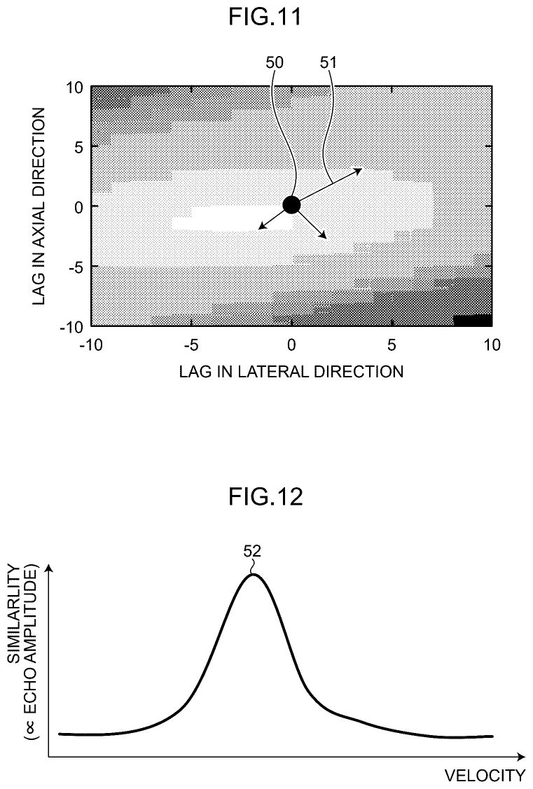

FIG. 11 and FIG. 12 are diagrams for explaining an example of processing executed by the ultrasonic diagnostic apparatus 1 according to the third embodiment using a speckle tracking method. FIG. 11 depicts a movement vector 51 from a local blood vessel location 50 in one of the power image data 21 to the search position in the other power image data 21 when a plurality of similarities are calculated. The normalization function 190c calculates the blood flow velocity by dividing the amount of movement (distance) indicated by the movement vector 51 by the time between frames. In this way, the amount of movement indicated by the movement vector 51 is converted into blood flow velocity.

The normalization function 190c then calculates an absolute velocity spectrum 52 illustrated in FIG. 12 based on the blood flow velocity and the error function (step S402). The absolute velocity spectrum 52 is a curve with similarity on the vertical axis and blood flow velocity on the horizontal axis. This blood flow velocity is the velocity of blood flow in the main direction obtained by the speckle tracking method. Therefore, there is no need to perform angular correction to the absolute velocity spectrum 52. Therefore, the absolute velocity spectrum 52 is information with good accuracy.

In the processing at each of steps S403, S404, and S108, the ultrasonic diagnostic apparatus 1 uses the absolute velocity spectrum 52 to execute processing similar to the processing at each of steps S106, S107, and S108 executed using the Doppler frequency spectrum 25 in the first embodiment. The absolute velocity spectrum 52 is, for example, an example of statistical information about blood flow.

The ultrasonic diagnostic apparatus 1 according to the third embodiment has been described above. According to the third embodiment, information about blood flow with good accuracy can be obtained in a non-contrast manner. In addition, according to the third embodiment, the Doppler frequency spectrum 25 itself does not need to be generated because the speckle tracking method is used.

### Fourth Embodiment

An image processing apparatus 60 according to a fourth embodiment will now be described. The image processing apparatus 60 performs processing similar to those of the ultrasonic diagnostic apparatus 1 described above. Therefore, according to the fourth embodiment, information about blood flow can be obtained in a non-contrast manner.

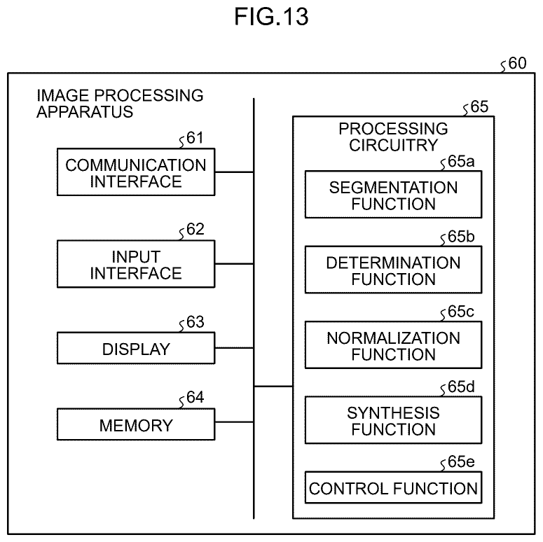

FIG. 13 is a diagram illustrating an example configuration of the image processing apparatus 60 according to the fourth embodiment.

As illustrated in FIG. 13, the image processing apparatus 60 includes a communication interface 61, an input interface 62, a display 63, a memory 64, and processing circuitry 65.

The communication interface 61 controls transmission and communication of various information and data transmitted and received between the image processing apparatus 60 and the ultrasonic diagnostic apparatus 1 connected to the image processing apparatus 60 by wired or wireless means. The communication interface 61 is connected to the processing circuitry 65. The communication interface 61 receives information and data transmitted by the ultrasonic diagnostic apparatus 1. In this case, the communication interface 61 transmits the received information and data to the processing circuitry 65. The communication interface 61 also receives information and data transmitted by the processing circuitry 65. In this case, the communication interface 61 transmits the received information and data to an external device. For example, the communication interface 61 is implemented by a network card, a network adapter, a network interface controller (NIC), or the like. For example, the communication interface 61 receives various data, such as reflected wave data and power image data 21 transmitted by the ultrasonic diagnostic apparatus 1, and transmits the received various data to the processing circuitry 65.

The input interface 62 accepts input operations for various instructions and various information from the user. The input interface 62 is connected to the processing circuitry 65. The input interface 62 converts the operations accepted from the user into electrical signals and transmits the electrical signals to the processing circuitry 65. For example, the input interface 62 is implemented by a trackball, a switch button, a mouse, a keyboard, a touchpad that accepts operations by touching the operation surface, a touchscreen that integrates a display screen and a touchpad, a non-contact input interface using an optical sensor, and a voice input interface. The input interface 62 herein is not limited only to those with physical operating components such as a mouse and a keyboard. For example, electrical signal processing circuitry that receives electrical signals corresponding to input operations from an external input device installed separately from the image processing apparatus 60 and transmits these electrical signals to the processing circuitry 65 is also an example of the input interface 62.

The display 63 displays various information and data. The display 63 is connected to the processing circuitry 65. The display 63 displays various information and data transmitted by the processing circuitry 65, under control by the processing circuitry 65. For example, the display 63 may be implemented by a display such as a liquid crystal display and a touch panel. The display 63 is, for example, an example of a display unit.

The memory 64 stores therein various data and computer programs. The memory 64 is connected to the processing circuitry 65. The memory 64 stores therein data transmitted by the processing circuitry 65, under control by the processing circuitry 65. For example, the memory 64 stores therein various data such as reflected wave data and power image data 21. The data stored in the memory 64 is read by the processing circuitry 65. For example, the memory 64 is implemented by a semiconductor memory element such as a random access memory (RAM) and a flash memory, a hard disk, or an optical disk.

The processing circuitry 65 controls the entire image processing apparatus 60. For example, the processing circuitry 65 executes various processing in response to instructions accepted from the user via the input interface 62.

As illustrated in FIG. 13, the processing circuitry 65 includes a segmentation function 65a, a determination function 65b, a normalization function 65c, a synthesis function 65d, and a control function 65e. Each of the processing functions 65a to 65e of the processing circuitry 65 performs processing similar to the processing executed by each of the processing functions 190a to 190d of the image processing circuitry 190 and the control circuitry 180 according to the first embodiment, using various data such as reflected wave data and power image data 21 stored in the memory 64. The segmentation function 65a has a function similar to the segmentation function 190a. The determination function 65b has a function similar to the determination function 190b. The normalization function 65c has a function similar to the normalization function 190c. The synthesis function 65d has a function similar to the synthesis function 190d. The control function 65e has a function similar to the function of the control circuitry 180. The segmentation function 65a is, for example, an example of a segmentation unit. The determination function 65b is, for example, an example of a determination unit. The normalization function 65c is, for example, an example of a normalization unit. The synthesis function 65d is, for example, an example of a synthesis unit. The control function 65e is, for example, an example of a display control unit or a control unit.

The processing circuitry 65 is implemented, for example, by a processor. In this case, each of the processing functions 65a to 65e described above is stored in the memory 64 in the form of a computer program (information processing program) executable by a computer. The processing circuitry 65 then reads each computer program stored in the memory 64 and executes the read computer program to implement the processing function corresponding to the computer program. In other words, the processing circuitry 65 has each processing function illustrated in FIG. 1 in a state in which each computer program is read.

The processing circuitry 65 may be configured as a combination of a plurality of independent processors, each processor executing a computer program to implement each processing function. The processing functions of the processing circuitry 65 may be distributed or integrated into single or a plurality of processing circuitry. The processing functions of the processing circuitry 65 may be implemented by a mixture of hardware such as circuitry and software. Although an example in which each computer program corresponding to each processing function is stored in the single memory 64 is described here, each computer program may be distributed and stored in a plurality of storage circuitry. For example, each computer program corresponding to each processing function may be distributed and stored in a plurality of storage circuitry, and the processing circuitry 65 may read and execute each computer program from the storage circuitry.

The image processing apparatus 60 according to the fourth embodiment has been described above. According to the fourth embodiment, information about blood flow can be obtained in a non-contrast manner, in the same manner as in the first embodiment.

The computer program to be executed by the processor is embedded in advance and provided in a read only memory (ROM), storage circuitry, or the like. The computer program may be recorded and provided as a file in a format that can be installed on these devices or in an executable format on a computer-readable non-transitory recording medium such as a compact disc (CD)-ROM, a flexible disk (FD), a CD-R (recordable), and a digital versatile disc (DVD). The computer program may be provided or distributed by being stored on a computer connected to a network such as the Internet and downloaded over the network. For example, the computer program includes modules including each of the processing functions described above. As actual hardware, the CPU reads and executes a computer program from a recording medium such as a ROM, and each module is loaded onto the main memory and generated on the main memory.

According to at least one embodiment and at least one modification described above, information about blood flow can be obtained in a non-contrast manner.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising processing circuitry configured to:
divide at least a part of an ultrasonic image into a plurality of regions;
determine whether each of the regions is a vascular region;
normalize statistical information about blood flow in a region determined to be a vascular region; and
synthesize the normalized statistical information.

2. The ultrasonic diagnostic apparatus according to claim 1, wherein
the processing circuitry
generates synthetic statistical information indicating a statistical distribution of vessel count with respect to blood flow velocity by synthesizing the normalized statistical information, and generates statistical information for display about blood flow velocity from the synthetic statistical information, and
displays the statistical information for display on a display.

3. The ultrasonic diagnostic apparatus according to claim 2, wherein
the processing circuitry
generates, as the statistical information for display, a statistical graph indicating a statistical distribution of vessel count with respect to blood flow velocity, and
displays the statistical graph on the display.

4. The ultrasonic diagnostic apparatus according to claim 2, wherein
the processing circuitry
calculates, as the statistical information for display, at least one of average flow velocity, standard deviation, mode, median, variance, and third-order or higher moment of blood flow velocities of a plurality of vessels, and
displays the calculated at least one of average flow velocity, standard deviation, mode, median, variance, and third-order or higher moment of blood flow velocities of the vessels on the display.

5. The ultrasonic diagnostic apparatus according to claim 1, wherein the processing circuitry calculates, as the statistical information, a spectrum indicating an absolute velocity at a local blood vessel location, using speckle tracking.

6. The ultrasonic diagnostic apparatus according to claim 1, wherein the processing circuitry calculates the statistical information by Fourier analysis in a frame direction of reflected wave data in the region determined to be a vascular region.

7. The ultrasonic diagnostic apparatus according to claim 1, wherein the processing circuitry calculates, as the statistical information, a Doppler spectrum of the region determined to be a vascular region.

8. The ultrasonic diagnostic apparatus according to claim 7, wherein
the Doppler spectrum indicates a relationship between echo intensity and blood flow velocity, and
the processing circuitry normalizes the Doppler spectrum based on echo intensity for each region determined to be a vascular region.

9. The ultrasonic diagnostic apparatus according to claim 1, wherein the processing circuitry performs angular correction to the statistical information based on a main direction of blood flow in the region determined to be a vascular region, and normalizes the statistical information subjected to angular correction.

10. The ultrasonic diagnostic apparatus according to claim 9, wherein the processing circuitry estimates the main direction of blood flow from a plurality of ultrasonic images adjacent in a frame direction of the region determined to be a vascular region, using a speckle tracking method or a multi-angle Doppler method.

11. The ultrasonic diagnostic apparatus according to claim 1, wherein the processing circuitry determines whether each of the regions is the vascular region by performing threshold processing pixel by pixel on a spatial distribution based on each of the regions.

12. The ultrasonic diagnostic apparatus according to claim 2, wherein the processing circuitry generates a plurality of pieces of the synthetic statistical information for a predetermined period of time and generates statistical information for display about blood flow velocity from new synthetic statistical information obtained by averaging the pieces of the synthetic statistical information.

13. The ultrasonic diagnostic apparatus according to claim 1, wherein the processing circuitry generates a blood flow image as the ultrasonic image from a plurality of pieces of reflected wave data collected along a time series by a transmit aperture synthesis method or a plane wave compound method.

14. An ultrasonic diagnostic apparatus comprising processing circuitry configured to:
acquire a vascular region by morphology analysis of a spatial distribution based on an ultrasonic image;
normalize statistical information about blood flow in the vascular region, and
synthesize the normalized statistical information.

15. The ultrasonic diagnostic apparatus according to claim 14, wherein
the statistical information is a Doppler spectrum,
the Doppler spectrum indicates a relationship between echo intensity and blood flow velocity, and
the processing circuitry
performs, for each vascular region, first normalization on the Doppler spectrum based on an area of the vascular region, and second normalization on the Doppler spectrum subjected to the first normalization based on echo intensity, and
synthesizes the Doppler spectrum subjected to the second normalization.

16. An image processing apparatus comprising processing circuitry configured to:
divide at least a part of an ultrasonic image into a plurality of regions;
determine whether each of the regions is a vascular region;
normalize statistical information about blood flow in a region determined to be a vascular region; and
synthesize the normalized statistical information.

17. A method comprising: by a computer,
dividing at least a part of an ultrasonic image into a plurality of regions;
determining whether each of the regions is a vascular region;
normalizing statistical information about blood flow in a region determined to be a vascular region; and
synthesizing the normalized statistical information.

18. A non-transitory computer readable medium comprising instructions that cause a computer to execute:
dividing at least a part of an ultrasonic image into a plurality of regions;
determining whether each of the regions is a vascular region;
normalizing statistical information about blood flow in a region determined to be a vascular region; and
synthesizing the normalized statistical information.
